(19) United States
(12) Patent Application Publication
(10) Pub. No.: US 2007/0160696 A1
(43) Pub. Date: Jul. 12, 2007

(54) Compositions and methods useful for prevention or treatment of respiratory illness

(75) Inventors: Jeffrey Warren Clymer, Mason, OH (US); Begonia Y. Ho, Cincinnati, OH (US); Mary Lynn Jump, Fairfield, OH (US); Amy Ann Walanski, Fairfield, OH (US); Claudine Killar Zukowski, Cincinnati, OH (US)

Correspondence Address: THE PROCTER & GAMBLE COMPANY;INTELLECTUAL PROPERTY DIVISION - WEST BLDG., WINTON HILL BUSINESS CENTER - BOX 412, 6250 CENTER HILL AVENUE, CINCINNATI, OH, 45224, US

(73) Assignee: The Procter & Gamble Company, Cincinnati, OH (US)

(21) Appl. No.: 11/330,299

(22) Filed: Jan. 11, 2006

(51) Int. Cl. A61K 36/906

(52) U.S. Cl. 424/756; 424/773

(57) ABSTRACT

Disclosed herein are compositions and methods useful for the treatment of respiratory illness in a mammal, such as wherein these compositions and methods are effective in preventing the onset of one or more of the symptoms of such respiratory illness or significantly mitigating the symptom if the mammal is already afflicted with such symptom. The compositions used in the methods herein include a first component selected from the group consisting of andrographis extracts, active components thereof, and mixtures thereof, and a second component selected from the group consisting of turmeric extracts, active components thereof, and mixtures thereof.

### FIELD OF THE INVENTION

The present invention is directed to compositions and methods useful for the treatment of respiratory illness, wherein these compositions and methods are effective in preventing the onset of one or more symptoms of respiratory illness in a mammal or significantly mitigating such a symptom if the mammal is already afflicted with such symptoms.

### BACKGROUND OF THE INVENTION

It is known that many different viruses and viral strains result in symptoms associated with respiratory illness. For example, the common cold is a complex syndrome that may be caused by any of over 200 antigenically different viruses found in several virus families. These families include rhinovirus, myxovirus, paramyxovirus, respiratory syncytial virus, adenovirus and coronavirus. Among the most important, with respect to the common cold, is rhinovirus. Pinpointing the specific cause of the illness is difficult and not practical since there are also a number of predisposing factors whose contribution to the manifestation of symptoms is not fully understood.

Regardless of the virus and associated factors leading to the onset of symptoms associated with respiratory illness, a number of remedies to alleviate these symptoms have been suggested. For example, the many cough/common cold products that are currently marketed typically contain one or more of the following actives: nasal decongestants such as pseudoephedrine or oxymetazoline, antihistamines such as doxylamine; antitussives such as dextromethorphan; expectorants such as guaifenesin; and anti-pyretics such as acetaminophen. In an attempt to improve existing common cold remedies, experts in the field have suggested several alternative pharmacotherapies and have conducted common cold trials to test their efficacy.

Despite the abundance of treatments known in the art, there remains a need to provide a consistent and effective method for prevention or treatment of respiratory illness. There also remains a need to provide compositions, particularly oral compositions, and methods, that are highly effective in the prevention or treatment of respiratory illness, such as those wherein the compositions provide anticholinergic or immunomodulatory activity.

### SUMMARY OF THE INVENTION

The present invention is directed to compositions and methods useful for the treatment of respiratory illness in a mammal, wherein these compositions and methods are effective in preventing the onset of one or more of the symptoms of such respiratory illness or significantly mitigating the symptom if the mammal is already afflicted with such symptom.

Surprisingly, the combination of andrographis extract, active components thereof, or mixture thereof, and a turmeric extract, active components thereof, or mixture thereof, provide an unexpected synergistic anticholinergic effect, which is highly beneficial in treatment of respiratory illness.

In one embodiment herein, the invention is directed to compositions comprising an andrographis extract, active components thereof, or mixture thereof and a turmeric extract, active components thereof, or mixture thereof.

In one embodiment herein, the invention is directed to methods of treating respiratory illness in a mammal in need of such treatment, comprising administering to the mammal a composition comprising an andrographis extract, active components thereof, or mixture thereof and a turmeric extract, active components thereof, or mixture thereof.

These and other aspects of the present invention are described in further detail herein.

### DETAILED DESCRIPTION OF THE INVENTION

Various documents including, for example, publications and patents, are recited throughout this disclosure. All such documents are hereby incorporated by reference.

All percentages and ratios are calculated by weight unless otherwise indicated. All percentages and ratios are calculated based on the total composition unless otherwise indicated.

Referenced herein are trade names for components including various ingredients utilized in the present invention. The inventors herein do not intend to be limited by materials under a certain trade name. Equivalent materials (e.g., those obtained from a different source under a different name or reference number) to those referenced by trade name may be substituted and utilized in the descriptions herein.

In the description of the invention various embodiments or individual features are disclosed. As will be apparent to the ordinarily skilled practitioner, all combinations of such embodiments and features are possible and can result in preferred executions of the present invention.

The compositions herein may comprise, consist essentially of, or consist of any of the elements as described herein.

While various embodiments and individual features of the present invention have been illustrated and described, various other changes and modifications can be made without departing from the spirit and scope of the invention. As will also be apparent, all combinations of the embodiments and features taught in the foregoing disclosure are possible and can result in preferred executions of the invention.

The term “safe and effective amount” of a component, composition, or like material as used herein is an amount that is effective for the treatment of respiratory illness in a mammal (preferably a human), without undue adverse side effects (such as toxicity, irritation, or allergic response), commensurate with a reasonable benefit/risk ratio when used in the manner of this invention. The specific “safe and effective amount” will, obviously, vary with such factors as the particular condition being treated, the physical condition of the treated mammal, the size and weight of the treated mammal, the duration of treatment, the nature of concurrent therapy (if any), the specific dosage form to be used, other components present in a given dosed composition, and the dosage regimen desired for the component or composition.

Compositions and Components Used in the Present Invention

In one embodiment, the compositions herein comprise a first and a second component, the first component comprising an andrographis extract, active components thereof, or mixture thereof and the second component comprising a turmeric extract, active components thereof, or mixture thereof. Without intending to be limited by theory, it has been found that upon oral administration, the compositions of the present invention induce immunomodulatory activity to counter viral infection and provide anticholinergic activity that treats respiratory illness, for example alleviation of a symptom such as nasal secretions and runny nose. Surprisingly, the combination of an andrographis extract and a turmeric extract provides an unexpected synergistic anticholinergic effect, which is highly beneficial in treatment of respiratory illness, including prevention or treatment of an existing symptom. As such, the present compositions are suitable for applications of preventing, reducing or eliminating the possibility of acquisition of such respiratory illness when confronted with a high-risk public environment including schools, office buildings, public transit, and public events.

Without being bound by theory, it is believed that the extract of andrographis extract, active components thereof or mixture thereof may provide, for example, enhanced immunomodulatory activity against virus, while the turmeric extract, active components thereof, or mixture thereof may provide anticholinergic activity to reduce, for example, symptoms of nasal secretions and runny nose. Surprisingly, that the present inventors have discovered that the combination of these components provides, as an example, enhanced anticholinergic activity over either component alone to result in improved treatment of respiratory illness.

The components of the present inventive compositions, as well as those components utilized in the methods herein, as described further below, are described as follows.

Andrographis Extract or Active Components Thereof

The present compositions comprise an andrographis extract, an active component thereof, or mixtures thereof. As used herein, the andrographis is a plant of the genus Andrographis, having a limited number of species within this genus largely present in Asia. Only a few of the species are medicinal. In one embodiment, the plant is of the species Andrographis paniculata, which may be referenced as Kalmegh in Ayurvedic medicine.

Andrographis paniculata is an annual herb. Andrographis paniculata has been used as a drug in traditional Siddha and Ayurvedic systems of medicine as well as in tribal medicine in India and some other countries for multiple clinical applications. The extract is reported to exhibit antityphoid and antifungal activities. This extract is also reported to exhibit antihepatotoxic, antibiotic, antimalarial, antihepatitic, antithrombogenic, antiinflammatory, and antipyretic properties, besides its general use as an immunostimulant. Andrographis paniculata and its principal components, andrographolides, are not believed to exhibit measurable anticholinergic activity when administered as a sole component.

In one embodiment, the composition comprises an andrographis extract. In this embodiment, the extract may be an extract of an aerial part of the plant (leaves, stems, or mixtures thereof). Without limitation by theory, it is believed that the leaves contain the highest amount of andrographolides, believed to be the most medicinally active phytochemicals in the plant.

More preferred extracts comprise one or more active components of andrographis, as described further below. Preparation of the andrographis extract may be performed using ethanol.

In another embodiment, the composition comprises an active component of andrographis. The active components of andrographis include a family of lactones, including diterpene lactones including but not limited to andrographolide, deoxyandrographolide, neoandrographolide, dehydroandrographolide, selenoandrographolide, homoandrographolide, andrographan, andrographon, andrographin, andrographosterin, 14-deoxy-11-oxoandrographolide, 14-deoxy-11,12-didehydroandrographolide, andrographiside, kalmeghin, and edelin lactone.

In one embodiment, the compositions may optionally comprise from about 1% to about 50%, alternatively from about 5% to about 40%, and alternatively from about 10% to about 20% andrographis extract, all by weight of the composition. In one embodiment, the compositions may optionally comprise from about 0.001% to about 10%, alternatively from about 0.01% to about 10%, alternatively from about 0.5% to about 5% by weight of the composition, and alternatively from about 1% to about 2% active component of andrographis, all by weight of the composition.

Turmeric Extract or Active Components Thereof

The present compositions comprise a turmeric extract, an active component thereof, or mixtures thereof. Turmeric is a bright yellow powder derived from the rhizomes of Curcuma longa. The aqueous extract of turmeric may be referred to as turmerin and the lipid extract of turmeric contains curcuminoids. Generally, without intending any limitation, the curcuminoids consist of about 75% curcumin, about 20% demethoxycurcumin and less than about 5% bis-demethoxycurcumin. The chemical name for curcumin is diferuloyl methane.

As such, the present compositions may comprise an active component of turmeric, such as a curcuminoid, for example, curcumin, demethoxycurcumin, or bis-demethoxycurcumin. In one embodiment, the active component is curcumin.

Surprisingly, the inventors have discovered that the turmeric extract or active components thereof possess surprisingly efficacious anticholinergic activity, particularly in combination with the andrographis extract or active components thereof (as described herein above).

In one embodiment, efficacy of the turmeric extract or active component thereof may be further enhanced through inclusion of an agent such as piperine, bromelain or lecithin.

In one embodiment, the compositions may optionally comprise from about 1% to about 75%, alternatively from about 10% to about 50%, and alternatively from about 20% to about 40% turmeric extract, all by weight of the composition. In one embodiment, the compositions may optionally comprise from about 1% to about 75%, alternatively from about 10% to about 50%, and alternatively from about 20% to about 40% active component of turmeric extract, all by weight of the composition.

Optional Components of the Present Compositions

In addition to the andrographis extract, active components and mixtures thereof, and the turmeric extract, active components, and mixtures thereof, the compositions herein may further comprise any of a variety of further optional components such as other active components or carrier materials.

Optional components suitable for use herein include materials such as binders, glidants, surfactants, lubricants, coating agents, disintegrants, chelating agents, preservatives, sweeteners, sensates, flavors, colorants, fragrances, and the like. Unless otherwise specified, the compositions may optionally comprise one or more given optional components at concentrations ranging from about 0.001% to about 99%, alternatively from about 0.01% to about 80%, alternatively from about 0.01% to about 50%, alternatively from about 0.01% to about 10%, all by weight of the composition.

Non-limiting examples of certain optional components are as follows.

Eleutherococcus senticosus or Active Component Thereof

The present compositions may optionally comprise Eleutherococcus, such as that of the species Eleutherococcus senticosus such as Eleutherococcus root, extracts thereof, active components thereof, or mixtures thereof. Eleutherococcus (Acanthopanax) senticosus has been referred to as Siberian ginseng. As used herein, the preferred common name now is eleuthero. Eleutherococcus is valued as an adaptogen and is believed to help combat fatigue. Other herbal extracts that are also thought to fight fatigue through stimulant adaptogenic effects are Rhodiola rosea and Schizandra chinensis.

In one embodiment, the composition comprises an eleutherococcus extract generally obtained from the root of the plant, or preparations of the root itself. Most preferred extracts comprise one or more active components of Eleutherococcus.

In another embodiment, the composition comprises an active component of eleutherococcus. In one embodiment, the active component is an eleutheroside.

In one embodiment, the compositions may optionally comprise from about 0.1% to about 20%, alternatively from about 0.1% to about 10%, and alternatively from about 0.1% to about 5% eleutherococcus extract, all by weight of the composition. Wherein the root is utilized, these levels may be increased by a factor of about 10 or about 20.

Pharmaceutically Acceptable Carriers and Other Optional Components

The compositions of the present invention may be administered orally as compositions comprising a pharmaceutically acceptable carrier system. Any pharmaceutically acceptable carrier in the form of a liquid, solid, or gas is suitable for the delivery of the compositions to prevent and treat common cold, influenza-like, and allergy symptoms.

Depending on the desired dose form of the composition and, where applicable, the delivery device to be used, the compositions of the present invention may optionally include a pharmaceutically acceptable carrier such as water, water-miscible solvents including ethanol, propylene glycol, polyethylene glycol, transcutol, glycerol, and other known or otherwise effective water-miscible solvents; liquid aerosol propellants; and mixtures thereof. In one embodiment, these carriers are isotonic with human plasma.

When the compositions of the present invention are administered using water as a pharmaceutically acceptable carrier, the water may optionally be purified or de-ionized water, and may be substantially free of organic impurities and/or meets the USP guidelines for purified water. The concentration of water utilized to formulate the compositions as a final product form for delivery to respiratory tract areas may range from about 40% to about 99.98%, alternatively from about 80% to about 99.95%, by weight of the final composition.

When the compositions of the present invention are administered using a solid pharmaceutically acceptable carrier, the carrier may be dosed as a powder, a capsule or tablet form. Pharmaceutically acceptable solid carriers can be added to provide aid in processing of the compositions, to aid in the consistency of the compositions, to provide for improved stability, to facilitate handling, for hygroscopicity benefits, and so forth. Pharmaceutically acceptable solid carrier materials include ingredients such as particulate and powder fillers, for example, a lactose powder, a sucrose powder and/or mixtures thereof.

As another non-limiting example, the composition may comprise a sweetener. Some natural and artificial sweeteners usable with the present invention include but are not limited to glucose, fructose, saccharine and its salts, sucrose, cyclamates, xylatols, acesulfame K, sucralose, and aspartame.

A specific nonlimiting example of another optional component suitable for use in the present invention in a liquid form include optional pH adjusting agents. Such optional pH adjusting agents include those normally associated with use in liquid compositions including compounds such as sodium bicarbonate, sodium phosphate, sodium hydroxide, ammonium hydroxide, sodium stannate, triethanolamine, sodium citrate, disodium succinate, and mixtures thereof. If present, the optional pH adjusting agents may be included at concentrations ranging from about 0.01% to about 5%, by weight of the composition.

Another specific nonlimiting example of an optional component suitable for use in the present invention includes chelating agents. Optional chelating agents useful in the compositions of the present invention include those that chelate transition metal ions such as iron, copper, zinc and other such metals. The optional chelating agents are stable and effective in non-aqueous and aqueous mediums, and at pH ranges between about 3 to about 6 in aqueous medium.

Nonlimiting examples of suitable optional chelating agents include physic acid, sodium and calcium salts of ethylene diamine tetraacetic acid (EDTA), tetrasodium EDTA, sodium hexametaphosphate (SHMP), di(hydroxyethyl)glycine, 8-hydroxyquinoline, and mixtures thereof.

Wherein the compositions of the present invention comprise one or more optional chelating agents, the chelating agents are included at concentrations ranging from about 0.001% to 10%, alternatively from about 0.005% to about 5%, alternatively from about 0.01% to about 2%, all by weight of the composition.

Additionally, the present invention may optionally include one or more antioxidant compounds to reduce or combat free radical formation. Some non-limiting examples of antioxidants usable in the present invention include vitamin E, BHT, BHA, benzoic acid, and ascorbic acid.

Other specific non-limiting examples of optional components suitable for use in the present invention include preservatives. One or more preservatives can optionally be included to prevent microbial contamination that can be attributed to dosing devices of the composition applied to the nose. Such optional preservatives include those normally associated with use in nasal compositions including benzalkonium chloride, chlorhexidine gluconate, phenyl ethyl alcohol, phenoxyethanol, benzyl alcohol, sorbic acid, thimerosal, phenylmercuric acetate, benzoates, parabens, sorbates, and mixtures thereof.

Method of Making

The compositions of the present invention may be prepared by any known or otherwise effective techniques suitable for providing a composition that provides a therapeutic benefit in the prevention and treatment of common cold, influenza- like, and allergy symptoms. The compositions are preferably formulated to comprise an andrographis extract and a turmeric extract, wherein these compositions may then be manufactured into final product forms of liquids, sprays, powders, capsules, tablets, or the like for oral ingestion and absorption to prevent or treat symptoms associated with respiratory illness.

As a non-limiting example, the following procedure may be utilized. The compositions exemplified herein below in Examples 1 and 2 may be prepared by adding together andrographis extract, turmeric extract, and the optional eleutherococcus extract, and optionally a filler material, blending the resulting mixture, optionally adding a lubricant and blending the resulting mixture. The final blend may then be encapsulated for example in gelatin capsules.

As another non-limiting example, the following procedure may be utilized. The compositions exemplified herein below in Example 3 may be prepared by adding together andrographis extract, turmeric extract, the optional eleutherococcus extract, and optionally a filler/binder material, a disintegrant material, blending the resulting mixture, optionally adding a lubricant and blending the resulting mixture. The final blend may then be compressed into tablets. The tablets may be optionally coated with a sugar or film-coating material.

Methods of the Present Invention

In a further embodiment, the present invention is directed to methods of treating a respiratory illness comprising orally administering a composition as described herein to a mammal in need of such treatment. As used herein, the term “treating”, with reference to the respiratory illness refers to preventing, alleviating a symptom, or curing. As used herein, the term “respiratory illness” encompasses a broad range of respiratory ailments, including viral infections such as influenza and common cold, as well as allergy. Respiratory illness may present as any of a variety of symptoms, such as runny nose, sneezing, rhinorrhea, nasal congestion, chest congestion, cough, pressure, headache, body ache, fever, sore throat, fatigue, chills, and the like. The mammal treated may be a human, or a companion animal such as a dog, cat or horse.

As used herein the term “orally administering” with respect to the mammal means that the mammal ingests or is directed to ingest, or does ingest, one or more of the present compositions. Wherein the human is directed to ingest the composition, such direct may be that which instructs and/or informs the human that use of the composition may and/or will provide relief from the respiratory illness (e.g., symptomatic relief, whether temporary or permanent) for example, relief from congestion. For example, such direction may be oral direction (e.g., through oral instruction from, for example, a physician, pharmacist, or other health professional), radio or television media (e.g., advertisement), or written direction (e.g., through written direction from, for example, a physician, pharmacist, or other health professional (e.g., scripts), sales professional organization (e.g., through, for example, marketing brochures, pamphlets, or other instructive paraphernalia), written media (e.g., internet, electronic mail, or other computer-related media)), and/or packaging associated with the composition (e.g., a label present on a container holding the composition). As used herein “written” means through words, pictures, symbols, and/or other visible or tactile descriptors. Such information need not utilize the actual words used herein, for example, “respiratory”, “illness”, or “mammal”, but rather use of words, pictures, symbols, tactile means, and the like conveying the same or similar meaning are contemplated within the scope of this invention.

Administration may be on an as-needed or as-desired basis, for example, once-monthly, once-weekly, or daily, including multiple times daily, for example, at least once daily, from one to about six times daily, from about two to about four times daily, or about three times daily.

The amount of composition administered may be dependent on a variety of factors, including the general quality of health of the mammal, age, gender, weight, or severity of symptoms.

In one embodiment herein, an oral composition is administered to the mammal, that provides in total dosage amounts, per dose, of from about 10 to about 1500 μg/kg body weight of the mammal, altneratively from about 50 to about 750 μg/kg body weight of the mammal, alternatively from about 100 to about 300 μg/kg body weight of the mammal, of total andrographolides.

In one embodiment herein, an oral composition is administered to the mammal, that provides in total dosage amounts, per dose, of from about 0.1 to about 50 μg/kg body weight of the mammal, altneratively from about 1 to about 30 μg/kg body weight of the mammal, alternatively from about 1 to about 20 μg/kg body weight of the mammal, alternatively from about 2.5 to about 10 μg/kg body weight of the mammal, of total curcuminoids.

The Eleutherococcus may be included at levels providing a dosage equivalent to eleutherococcus root of from about 0.1 to about 30 mg/kg body weight, alternatively from about 1 to about 20 mg/kg body weight, and alternatively from about 2.5 to about 10 mg/kg body weight of the mammal.

### EXAMPLES

The following examples further describe and demonstrate embodiments within the scope of the present invention. The examples are given solely for the purpose of illustration and are not to be construed as limitations of the present invention, as many variations thereof are possible without departing from the spirit and scope of the invention. All exemplified concentrations are weight-weight percents, unless otherwise specified.

Turmeric extract may be obtained from Sabinsa Corporation, Piscataway, N.J. Eleutherococcus and Andrographis extracts may be obtained from Dansk Droge, Denmark.

EXAMPLE 1

| Component | Amount per Capsule | |
|---|---|---|
| Andrographis paniculata extract | 51.0 | mg* |
| Turmeric extract | 166.7 | mg** |
| Eleutherococcus senticosus extract | 7.2 | mg*** |
| Piperine | 1.2 | mg |
| Microcrystalline Cellulose, Avicel PH 200 | 171.9 | mg |
| Magnesium Stearate | 2.0 | mg |

*51.0 mg Andrographis paniculata containing 5 mg andrographilides.
**166.7 mg Turmeric extract containing 158.3 mg curcuminoids.
***7.2 mg Eleutherococcus senticosus extract, equivalent to 120 mg Eleutherococcus senticosus root.

The andrographis, turmeric, eleutherococcus, piperine and cellulose powders are mixed together. The magnesium stearate is then added and the entire blend is mixed. The resulting powder blend is dispensed into capsules containing 400 mg each. Dosage is four capsules taken three times daily.

EXAMPLE 2

| Component | Amount per Capsule | |
|---|---|---|
| Andrographis paniculata extract | 102.0 | mg* |
| Turmeric extract | 333.3 | mg** |
| Eleutherococcus senticosus extract | 14.4 | mg*** |
| Piperine | 2.4 | mg |
| Microcrystalline Cellulose, Avicel PH 200 | 144.9 | mg |
| Magnesium Stearate | 3.0 | mg |

*102 mg Andrographis paniculata containing 10 mg andrographilides.
**333.3 mg Turmeric extract containing 316.7 mg curcuminoids.
***14.4 mg Eleutherococcus senticosus extract, equivalent to 240 mg Eleutherococcus senticosus root.

The andrographis, turmeric, eleutherococcus, piperine and cellulose powders are mixed together. The magnesium stearate is then added and the entire blend is mixed. The resulting powder blend is dispensed into capsules containing 600 mg each. Dosage is two capsules taken three times daily.

EXAMPLE 3

| Component | Amount per Tablet | |
|---|---|---|
| Andrographis paniculata extract | 102.0 | mg* |
| Turmeric extract | 333.3 | mg** |
| Eleutherococcus senticosus extract | 14.4 | mg*** |
| Piperine | 2.4 | mg |
| Povidone | 18.0 | mg |
| Croscarmellose, sodium | 12.0 | mg |
| Microcrystalline Cellulose, Avicel PH 200 | 114.9 | mg |
| Magnesium Stearate | 3.0 | mg |

*102 mg Andrographis paniculata containing 10 mg andrographilides.
**333.3 mg Turmeric extract containing 316.7 mg curcuminoids.
***14.4 mg Eleutherococcus senticosus extract, equivalent to 240 mg Eleutherococcus senticosus root.

The andrographis, turmeric, eleutherococcus, piperine, povidone, cellulose and half the croscarmellose sodium are mixed together with a small amount of water until granulation occurs. The granulation is oven-dried to remove the water, and the blend is milled. The remaining half of the croscarmellose sodium and the magnesium stearate is then added and the entire blend is mixed. The resulting powder blend is compressed into tablets containing 600 mg each. The tablets may be optionally coated with sugar or film coating. Dosage is two capsules taken three times daily.

What is claimed is:

1. A method of treating a respiratory illness in a mammal in need of such treatment, comprising administering to the mammal a safe and effective amount of a composition comprising:
(a) a first component selected from the group consisting of andrographis extracts, active components thereof, and mixtures thereof; and
(b) a component selected from the group consisting of turmeric extracts, active components thereof, and mixtures thereof.

2. The method of claim 1 wherein said composition further comprises a component selected from the group consisting of: eleutherococcus extracts, eleutherococcus root, active components thereof, and mixtures thereof; rhodiola rosea, extracts, active components thereof, and mixtures thereof; and schizandra chinensis extracts, active components thereof, and mixtures thereof; and mixtures thereof.

3. The method of claim 1 wherein said first component provides a dosage of total andrographolides of from about 10 to about 1500 μg/kg body weight of the mammal.

4. The method of claim 3 wherein said first component provides a dosage of total andrographolides of from about 50 to about 750 μg/kg body weight of the mammal.

5. The method of claim 4 wherein said first component provides a dosage of total andrographolides of from about 100 to about 300 μg/kg body weight of the mammal.

6. The method of claim 1 wherein said second component provides a dosage of total curcuminoids of from about 0.1 to about 50 mg/kg body weight of the mammal.

7. The method of claim 6 wherein said second component provides a dosage of total curcuminoids of from about 1 to about 30 mg/kg body weight of the mammal.

8. The method of claim 7 wherein said second component provides a dosage of total curcuminoids of from about 5 to about 20 mg/kg body weight of the mammal.

9. The method of claim 2 wherein said eleutherococcus extract, eleutherococcus root, active components thereof, and mixtures thereof provides a dosage equivalent to eleutherococcus root of from about 0.1 to about 30 mg/kg body weight of the mammal.

10. The method of claim 9 wherein said eleutherococcus extract provides a dosage equivalent to eleutherococcus root of from about 1 to about 20 mg/kg body weight of the mammal.

11. The method of claim 10 wherein said eleutherococcus extract provides a dosage equivalent to eleutherococcus root of from about 2.5 to about 10 mg/kg body weight of the mammal.

12. The method of claim 1 wherein said composition is administered in a form selected from the group consisting of: powders, capsules, tablets, liquids and sprays.

13. The method of claim 1 wherein said composition is administered from one to about six times daily.

14. The method of claim 13 wherein said composition is administered from about two to about four times daily.

15. The method of claim 14 wherein said composition is administered about three times daily.

16. The method of claim 1 wherein said composition further comprises a component selected from the group consisting of: piperine, bromelain, lecithin and mixtures thereof.

17. A composition comprising:
(a) a first component selected from the group consisting of andrographis extracts, active components thereof, and mixtures thereof; and
(b) a second component selected from the group consisting of turmeric extracts, active components thereof, and mixtures thereof.

18. The composition of claim 17 further comprising a component selected from the group consisting of eleutherococcus root, eleutherococcus extracts, active components thereof, and mixtures thereof; rhodiola rosea, active components thereof, and mixtures thereof; and schizandra chinensis, active components thereof, and mixtures thereof; and mixtures thereof.

19. The composition of claim 17 wherein said first component comprises from about 0.1% to about 10% total andrographolides, by weight of said composition.

20. The composition of claim 17 wherein said first component comprises from about 0.5% to about 5% total andrographolides, by weight of said composition.

21. The composition of claim 20 wherein said first component comprises from about 1% to about 2% total andrographolides, by weight of said composition.

22. The composition of claim 17 wherein said first component comprises an andrographolide selected from the group consisting of: diterpene lactones, andrographolide, deoxyandrographolide, neoandrographolide, dehydroandrographolide, selenoandrographolide, homoandrographolide, andrographan, andrographon, andrographosterin, 14-deoxy-11-oxoandrographolide, 14-deoxy-11,12-didehydroandrographolide, andrographiside, edelin lactone, and mixtures thereof.

23. The composition of claim 17 wherein said second component comprises from about 1% to 75% total curcuminoids, by weight of said composition.

24. The composition of claim 23 wherein said second component comprises from about 10% to about 50% total curcuminoids, by weight of said composition.

25. The composition of claim 24 wherein said second component comprises from about 20% to about 40% curcuminoids, by weight of said composition.

26. The composition of claim 17 wherein said second component comprises curcumin.

27. The composition of claim 17 wherein said composition is in a form selected from the group consisting of: powders, capsules, tablets, liquids and sprays.

28. The composition of claim 17 further comprising a component selected from the group consisting of: piperine, bromelain, lecithin and mixtures thereof.

29. The composition of claim 17 further comprising a component selected from the group consisting of: binders, glidants, surfactants, lubricants, coating agents, disintegrants, chelating agents, preservatives, sweeteners, sensates, flavors, colorants, fragrances, and mixtures thereof.

30. The composition of claim 17 comprising from about 40% to about 99.98% water, by weight of said composition.

31. The composition of claim 30 comprising from about 80% to about 99.95% water, by weight of said composition.

32. The composition of claim 31 further comprising from about 0.01% to about 5% of a pH adjusting agent, by weight of said composition.

33. The composition of claim 17 further comprising a chelating agent selected from the group consisting of: physic acid, sodium and calcium salts of ethylene diamine tetraacetic acid (EDTA), tetrasodium EDTA, sodium hexametaphosphate (SHMP), di(hydroxyethyl)glycine, 8-hydroxyquinoline, and mixtures thereof.
